(19) United States
(12) Patent Application Publication
(10) Pub. No.: US 2001/0038852 A1
(43) Pub. Date: Nov. 8, 2001

(54) Solid oral dosage forms with delayed release of active ingredient and high mechanical stability

(76) Inventors: Karl Kolter, Limburgerhof (DE); Michael Schonerr, Frankenthal (DE); Hermann Ascherl, Dirmstein (DE)

Correspondence Address: Herbert B. Keil, KEIL & WEINKAUF, 1101 Connecticut Ave., N. W., Washington, DC, 20036, US

(21) Appl. No.: 09/811,546

(22) Filed: Mar. 20, 2001

(30) Foreign Application Priority Data
Mar. 29, 2000 (DE) 10015479.4

(51) Int. Cl. A61K 9/20; A61K 9/28; A61K 9/14

(52) U.S. Cl. 424/465; 424/486

(57) ABSTRACT

The present invention relates to oral dosage forms with delayed release of active ingredient and high mechanical stability, comprising

a) one or more active ingredients

b) a formulated mixture of polyvinyl acetate and polyvinylpyrrolidone

c) water-soluble polymers or low or high molecular weight lipophilic additives

d) and other conventional excipients, and to the use and production thereof.

The present invention relates to solid oral dosage forms with delayed release of active ingredient and, at the same time, high mechanical stability, comprising besides a preformulated mixture of polyvinyl acetate and polyvinylpyrrolidone also other water-soluble polymers or lipophilic additives.

Slow release dosage forms continue to increase in importance on the one hand because the frequency of administration can be reduced, and on the other hand because they lead to a reduction in the variations in blood levels. The lower maximum blood level may reduce the severity of dose-dependent side effects and thus improve the tolerability for example in the case of drugs. The higher minimum plasma concentration increases the efficacy in particular of active ingredients for which the concentration should not fall below a particular threshold.

The desired protracted/controlled release of active ingredient can be effected by embedding the active ingredient in an inert matrix. Such (slow release) matrix tablets are usually produced by direct compression of the appropriate powder mixture or by previous granulation with subsequent compression. A further possibility for producing single-dose solid matrix shaped articles is provided by the extrusion process. After the compression or extrusion, the plastic composition (“matrix release-slowing means”) forms the porous coherent matrix in which the active ingredient(s) are homogeneously dispersed. The matrix formers (“matrix release-slowing means”) suitable for this purpose must meet the physicochemical requirements necessary for the appropriate processing technology (especially direct tableting), including good flowability and good compressibility.

The direct tableting process is a relatively simple, low-cost and time-saving process especially for producing drug forms and thus offers the pharmaceutical industry many advantages. In addition, direct tableting can be used to process even heat- and/or moisture-sensitive active ingredients.

The general requirements to be met by an excipient for direct tableting to produce slow-release matrix tablets are accordingly:

good flowability

great plastic deformability

little tendency to desegregation in the tableting mixture

formation of a matrix which is sufficiently mechanically stable for storage, transport and use

good release-slowing potential

release slowing independent of pH, ionic strength, mechanical stress

inert toward all active ingredients hydroxypropylmethylcellulose (Methocel®), which is the excipient employed to date most frequently for matrix tablets, shows the distinct disadvantage of poor flowability, low plasticity and poor compressibility.

Other excipients customary for matrix release slowing are, for example, hydroxypropylcellulose, xanthan and alginic acid. The following problems are evident overall on use of the excipients customary to date for matrix release slowing:

poor flowability

poor compressibility

tendency to stick

unfavorable effect on the active ingredient release profile through influences such as pH, ionic strength and mechanical stress etc.

batch variability with associated change in the product properties, especially with products of natural origin.

Tablets ought to be very mechanically stable because, otherwise, abrasion and breakage occur during further processing, for example during coating and packaging.

Assessment of the erosion stability of matrix slow-release formulations is important in as much as, for example, the peristalsis of the gastrointestinal tract may crucially influence the release characteristics. Particularly in the case of Methocel® with a swelling-controlled release-slowing matrix it would be possible for the swollen polymer layers to be abraded off uncontrollably, for example through friction with food constituents, which is contradictory to controlled matrix release slowing. An in vitro/in vitro correlation is thus extremely doubtful.

Inert matrix formers such as, for example, ethylcellulose, ammoniomethacrylate copolymer (Eudragit® RS or RL), stearyl alcohol and stearic acid likewise show numerous disadvantages such as poor flowability, poor compressibility, tendency to stick, active ingredient release influenced by changes in pH, and batch variability. An additional point is that, due to the high lipophilicity of some of these substances, active ingredients are in part completely enclosed in the matrix, leading to incomplete release of the total dose. This is unacceptable especially on use for drugs.

The formulated mixture of polyvinyl acetate and polyvinylpyrrolidone is, because it is an intimate mixture of a lipophilic with a hydrophilic polymer, more suitable for release slowing than are the abovementioned substances. Combinations of this type are described in U.S. Pat. No. 5,490,990.

Medicinal substance matrices based on a formulated mixture of polyvinyl acetate and polyvinylpyrrolidone gradually form, during passage through the stomach and intestines, fine pores through which the medicinal substance slowly diffuses out. The inert excipient matrix free of active ingredient is then excreted unchanged with the feces. This means that release of the active ingredient takes place substantially independent of external factors such as degree of filling of the stomach, intestinal motility etc. The diffusion-controlled release from such matrices can be described mathematically by the following equation:

$$Q = \sqrt{\frac{D \cdot ϵ}{τ} \pm ( 2 \cdot A \pm ϵ \cdot {C}_{S} ) \cdot {C}_{S}} \cdot \sqrt{t}$$

The formulated mixture of polyvinyl acetate and polyvinylpyrrolidone combines great mechanical stability with, at the same time, good slowing of release. The excellent flow properties and the high plasticity make it possible to process tableting mixtures which are otherwise critical. As synthetic product, the disadvantages of a natural product, such as variations in product quality due to batch inhomogeneity, do not of course apply.

Adjustment of the release of active ingredient must in principle take place individually for each active ingredient because it must be based on the pharmacological, biochemical and physicochemical properties of the active ingredient and the desired duration of action. It is known that release of active ingredient can be modified by increasing or decreasing the proportion of the formulated mixture of polyvinyl acetate and polyvinylpyrrolidone. This possibility of variation does not lead to a satisfactory result for diverse active ingredients. In addition, the size of a tablet is always altered thereby.

For active ingredients of low solubility a quite small amount of the formulated mixture of polyvinyl acetate and polyvinylpyrrolidone would suffice for release slowing, but the variations in the release from tablet to tablet are quite large because the matrix structure is subject to chance variations, and the mechanical stability of the tablet is poor. Incorporation of a larger amount of the formulated mixture of polyvinyl acetate and polyvinylpyrrolidone would be desirable.

For other active ingredients, the initial release is somewhat too rapid to be satisfactory, because release from nonswelling matrices obeys the “root t law”. It would therefore be desirable to reduce this rapid initial release through admixtures to the tablet formula without losing the advantages of the polyvinyl acetate/polyvinylpyrrolidone matrix.

With active ingredients which are very soluble in water the release from a polyvinyl acetate/polyvinylpyrrolidone matrix is often quite fast, and large amounts of polyvinyl acetate/polyvinylpyrrolidone are required, which greatly increase the size of the form and make it difficult to swallow. Slowing of release of such substances is at present possible only poorly with this release-slowing means.

Possibilities for adjusting these release profiles with retention of the matrix and the mechanical stability have not previously been disclosed.

It is an object of the present invention to develop a solid oral dosage form with delayed release of active ingredient and, at the same time, high mechanical stability.

We have found that this object is achieved by oral dosage forms with delayed release of active ingredient and high mechanical stability comprising

a) one or more active ingredients

b) a formulated mixture of polyvinyl acetate and polyvinylpyrrolidone

c) water-soluble polymers or low or high molecular weight lipophilic additives

d) and other conventional excipients.

The dosage forms are preferably employed for active pharmaceutical ingredients. However, they can also be employed for any other active ingredient for which delayed release is desired.

It is possible by the addition of water-soluble polymers or lipophilic additives to vary the release within almost any limits with, at the same time, good flowability of the tableting mixture, and great hardness and low friability of the tablets. It is possible by adding low-viscosity, nonswelling water-soluble polymers such as polyvinyl alcohols, polyethylene glycols, polyoxyethylene/polyoxypropylene block copolymers, polyvinylpyrrolidones and derivatives, vinyl acetate/vinylpyrrolidone copolymers, preferably polyethylene glycols, polyvinylpyrrolidones, vinyl acetate/vinylpyrrolidone copolymers or maltodextrins, to increase the rate of release of active ingredient.

These additives are employed in concentrations of from 1 to 40%, preferably from 2 to 30%, based on the total weight of the tablet. This is necessary for very low-dose active ingredients, where the amount of formulated mixture of polyvinyl acetate and polyvinylpyrrolidone needed to construct the matrix causes excessive slowing of release. This also applies to active ingredients of low solubility, for which although small amounts of release-slowing means lead to delayed release, the construction of the matrix is incomplete and subject to great variations, and the mechanical stability of the tablets is inadequate. This is especially the case when the active ingredient is difficult to compress.

Nor is it possible to improve decisively the poor flowability of the active ingredient by the small amount of formulated mixture of polyvinyl acetate and polyvinylpyrrolidone. Increasing the proportion of release-slowing means improves these properties but then leads to an excessive delay in release. The water-soluble nonswelling polymer increases the rate of release and stabilizes it in relation to external influences. The reproducibility is also very much better. Conventional tableting excipients such as lactose, calcium phosphates, sorbitol, mannitol, microcrystalline cellulose or starch are unable or insufficiently able to do this. It is probable that an interaction of the water-soluble polymer with a formulated mixture of the polymers polyvinyl acetate and polyvinylpyrrolidone leads to the very stable and reproducible release which is independent of the pressure for compression. The hardness of the tablets and the friability also show excellent values, which are often in fact better than without admixture of water-soluble polymers.

The friability should be less than 3%, preferably less than 1.5%, particularly preferably less than 1%.

Water-soluble but swelling, high-viscosity polymers surprisingly lead to slower release. It would have been expected that the inert matrix would be destroyed by the swelling polymer, and the active ingredient would be released more rapidly. The fact that this does not occur probably derives from the great elasticity of the formulated mixture of polyvinyl acetate and polyvinylpyrrolidone. The high-viscosity solution of the water-soluble, swelling polymer which forms in the pores of the matrix blocks them and thus slows down the diffusion of the active ingredient to the outside. The slowing of release is often greater than through the two components on their own. A synergistic effect is present. An additional point is that the initial release is also reduced through gel formation on the surface, and the release profile is thus “linearized”. The mechanical properties of the tablet remain at a very high level.

Water-soluble swelling polymers which can be employed are: alginates, pectins, galactomannans, carrageenans, dextran, curdlan, pullulan, gellan, chitin, gelatin, xanthans, hemicelluloses, cellulose derivatives such as methylcellulose, hydroxypropylmethylcellulose, hydroxypropylcellulose, hydroxyethylcellulose, methylhydroxyethylcellulose, carboxymethylcellulose, starch derivatives such as carboxymethylstarch, degraded starch, polyacrylic acid, polymethacrylic acid, acrylic acid/methacrylic acid copolymers. Possible salts of these substances are likewise included.

These additives are employed in concentrations of from 1 to 40%, preferably from 2 to 30%, based on the weight of the tablet.

The release-slowing effect can also be enhanced by fine-particle lipophilic additives. In this case, these additives infiltrate into the pores and channels of the matrix of polyvinyl acetate and polyvinylpyrrolidone and block them. It is important for the substances to be employed in small particle size because they display only a slight or no effect in coarse form. Lipophilic additives which can be used are both polymers and low molecular weight compounds. The polymers are, however, preferred.

These additives include: cellulose derivatives such as ethylcellulose, cellulose acetate, cellulose acetate phthalate, cellulose acetate succinate, hydroxypropylmethylcellulose acetate phthalate, hydroxypropylmethylcellulose acetate succinate, acrylic ester/methacrylic ester copolymers, especially methyl methacrylate/ethyl acrylate copolymers, ammoniomethacrylate copolymers type A and type B, methacrylic acid/acrylic ester copolymers, in particular methacrylic acid/ethyl acrylate copolymers, fatty alcohols such as stearyl alcohol, fatty acids such as stearic acid, fatty acid esters and fatty alcohol esters, glycerides, waxes, lecithin.

These additives are employed in concentrations of from 1 to 40%, preferably from 2 to 30%, based on the total weight of the tablet.

The formulated mixture of polyvinyl acetate and polyvinylpyrrolidone is present in the preparations according to the invention in concentrations of from 10 to 80%, preferably from 20 to 60%.

The ratio of polyvinyl acetate and polyvinylpyrrolidone in the formulated mixture is between 6:4 and 9:1. Ratios which are not within this range do not show the desired effect in relation to slowing of release and mechanical properties.

The dosage forms according to the invention comprise oral dosage forms such as tablets, extrudates, pellets or granules.

They can be produced by direct compression, extrusion, melt extrusion, pelleting or compaction.

Dry granulation processes and wet granulation processes can also be used.

Smaller shaped articles such as, for example, pellets or microtablets can also be introduced into capsules.

It is, of course, also possible to employ other conventional tableting excipients, for example binders, extenders/fillers, disintegrants, lubricants, flow regulators, dyes, stabilizers such as antioxidants, wetting agents, preservatives, release agents, flavorings and sweeteners.

Lubricants which can be used are stearates of aluminum, calcium, magnesium and tin, and magnesium silicate, silicones and the like.

Examples of possible flow regulators are talc or colloidal silica.

The binder is, for example, microcrystalline cellulose.

Disintegrants can be crosslinked polyvinylpyrrolidone or crosslinked sodium carboxymethylstarch. Stabilizers can be ascorbic acid or tocopherol.

Fillers which can be added are, for example, inorganic fillers such as oxides or magnesium, aluminum, silicon, titanium carbonate or calcium carbonate, calcium phosphates or magnesium phosphates or organic fillers such as lactose, sucrose, sorbitol, mannitol.

Examples of dyes are iron oxides, titanium dioxide, triphenylmethane dyes, azo dyes, quinoline dyes, indigotine dyes, carotenoids for coloring the dosage forms, opacifying agents such as titanium dioxide or talc in order to reduce the transparency to light and to save on dyes.

The dosage forms according to the invention may contain any active ingredient for which delayed release is desired.

The active ingredients preferably employed are food supplements or additives, vitamins, minerals or trace elements, but particularly preferably active pharmaceutical ingredients.

Pharmaceutical formulations of the abovementioned type can be obtained by processing the claimed compounds with active pharmaceutical ingredients by conventional methods and with use of known and novel active ingredients. The active ingredients may moreover come from any area of indications.

Examples which may be mentioned here are the following:

benzodiazepines, antihypertensives, vitamins, cytostatics, anesthetics, neuroleptics, antidepressants, antibiotics, antimycotics, fungicides, chemotherapeutics, urologicals, platelet aggregation inhibitors, sulfonamides, spasmolytics, hormones, immunoglobulins, sera, thyroid therapeutics, psychopharmaceuticals, antiparkinson agents and other antihyperkinetics, ophthalmologicals, neuropathy products, calcium metabolism regulators, muscle relaxants, lipid-lowering agents, liver therapeutics, coronary agents, cardiac agents, immunotherapeutics, regulatory peptides and their inhibitors, hypnotics, sedatives, gynecologicals, antigout agents, fibrinolytics, enzyme products and transport proteins, enzyme inhibitors, emetics, perfusion promoters, diuretics, diagnostics, corticoids, cholinergics, biliary therapeutics, antiasthmatics, bronchospasmolytics, beta-receptor blockers, calcium channel blockers, ACE inhibitors, arteriosclerosis remedies, antiinflammatory agents, anticoagulants, antihypotensives, antihypoglycemics, antifibrinolytics, antiepileptics, antiemetics, antidotes, antidiabetics, antiarrhythmics, antianemics, antiallergics, anthelmintics, analgesics, analeptics, aldosterone antagonists, weight-reducing agents.

The tablet shape may be varied within wide limits. Thus, biconvex, biplanar, round or polygonal tablets can be produced, as well as oblong or football shapes. The upper limit on size is determined by the swallowability, while the lower limit is determined by machine design limits. Conventional tablet sizes are between 1 and 16 mm, preferably between 2 and 13 mm, in diameter.

It is also possible to produce two-layer or multilayer tablets in which one layer contains the complete dose of active ingredient or at least has a very large active ingredient content, whereas the other layer has a very large content of the polyvinyl acetate/vinylpyrrolidone combination. It is possible in this way specifically to influence active ingredient release additionally. It is even possible on use of two or more active ingredients to release these at different rates by incorporating them entirely or for the most part separately in individual layers.

A particular embodiment is the production of press-coated tablets in which the core has a very large active ingredient content or may even contain the complete amount of active ingredient, whereas the covering consists to a large extent of the polyvinyl acetate/polyvinylpyrrolidone combination. This produces a great slowing of release. This form is particularly suitable for active ingredients which are very soluble in water and are intended to be released very slowly.

The tablets according to the invention can also be produced by melt extrusion and subsequent calendering.

The tablets can be provided in a conventional way with a film coating. This coating may be soluble in water, and then it merely serves to improve the visual appearance or mask an unpleasant odor or taste, but it may also be insoluble in water, and then is used to reduce release of active ingredient further. This is necessary if a very long duration of action is desired. It is possible in principle to employ all pharmaceutically approved coating materials, for example hydroxypropylmethylcellulose (Pharmacoat 603 or 606, supplied by Shin-Etsu), hydroxypropylcellulose, ethylcellulose, cellulose acetate phthalate, ammoniomethacrylate copolymer (USP), methacrylic acid copolymer type C (USP), butyl methacrylate/2-dimethylaminoethyl methacrylate/methyl methacrylate copolymer, polyvinyl acetate, polyvinylpyrrolidone.

The following examples are intended to explain the invention in detail without, however, restricting it thereto.

### EXAMPLE 1

Caffeine tablets with copolyvidone (Kollidon® VA 64)

Tableting mixture (A) consisting of 320 g of caffeine and 320 g of a formulated mixture of polyvinyl acetate and polyvinylpyrrolidone in the ratio 8:2 (=Kollidon® SR) and 3.2 g of Mg stearate; tableting mixture (B) consisting of 320 g of caffeine and 320 g of a formulated mixture of polyvinyl acetate and polyvinylpyrrolidone in the ratio 8:2, 80 g of Kollidon® VA 64 (copolymer of vinyl acetate and vinylpyrrolidone in the ratio 6:4) and 3.6 g of Mg stearate; tableting mixture (C) consisting of 320 g of caffeine and 360 g of a formulated mixture of polyvinyl acetate and polyvinylpyrrolidone in the ratio 8:2, 160 g of Kollidon® VA 64 and 4.2 g of Mg stearate;

Sieving of the individual powder ingredients through an 800 μm sieve, mixing in a Turbula mixer for 10 minutes. The respective tablets (10 mm, round, biplanar with beveled edge) were compressed in an eccentric press (Korsch EK0) under a pressure of 18 kN.

Determination of the hardness with a Kramer tablet tester (HAT-TMB), friability in an Erweka Friabilator; release test by USP XXIV method in an Erweka DT80 release apparatus, paddle method, 50 rpm, 0 to 2 h in 0.08 N HCl medium, the changed to pH 6.8 with phosphate buffer solution.

TABLE 1
Composition of the tablet batches [mg]:

| Batch: | A | B | C |
|---|---|---|---|
| Caffeine | 160 | 160 | 160 |
| Kollidon SR | 160 | 160 | 180 |
| Kollidon VA64 | — | 40 | 80 |
| Mg stearate | 1.6 | 2 | 1.8 |
| Hardness [N] | 295 | 325 | >325 |
| Friability [%] | 0.01 | <0.01 | <0.01 |

TABLE 2

| | Active ingredient release [%] | | |
|---|---|---|---|
| Time | K.SR 160 mg | K.SR/K.VA 64 | K.SR/K.VA 64 |
| [h] | [A] | 160/40 mg [B] | 160/80 mg [C] |
| 0 | 0 | 0 | 0 |
| 0.5 | 10.9 | 15.2 | 17.5 |
| 1 | 16.9 | 21.6 | 22.9 |
| 1.5 | 20.7 | 25.4 | 28.0 |
| 2.19 | 24.4 | 29.5 | 32.0 |
| 3 | 29.7 | 35.2 | 37.8 |
| 4 | 33.9 | 38.7 | 41.9 |
| 6 | 40.3 | 44.8 | 51.7 |
| 8 | 46.1 | 51.4 | 61.0 |
| 12 | 55.8 | 64.4 | 74.3 |
| 16 | 64.4 | 72.7 | 83.8 |

Addition of Kollidon® VA 64 increases the rate of release and improves the mechanical properties.

### EXAMPLE 2

Caffeine tablets with hydroxypropylmethylcellulose (Methocel® K 100 M)

Tableting mixture (A) cf. Ex. 1. Tableting mixture (D) consisting of 320 g of caffeine and 320 g of a formulated mixture of polyvinyl acetate and polyvinylpyrrolidone in the ratio 8:2 (=Kollidon® SR), 20 g of Methocel® 100M and 3.3 g of Mg stearate; tableting mixture (E) consisting of 320 g of caffeine and 20 g of Methocel K 100 M and 1.7 g of Mg stearate.

Sieving of the individual powder ingredients through an 800 μM sieve, mixing in a Turbula mixer for 10 minutes. The respective tablets (10 mm, round, biplanar with beveled edge) were compressed in an eccentric press (Korsch EK0) under a pressure of 18 kN.

Determination of the hardness with a Kramer tablet tester (HAT-TMB), friability in an Erweka Friabilator; release test by USP XXIV method in an Erweka DT80 release apparatus, paddle method, 50 rpm, 0 to 2 h in 0.08 N HCl medium, then changed to pH 6.8 with phosphate buffer solution.

TABLE 3
Composition of the tablet batches [mg]:

| Batch: | A | D | E |
|---|---|---|---|
| Caffeine | 160 | 160 | 160 |
| Kollidon SR | 160 | 160 | — |
| Methocel K100M | — | 10 | 10 |
| Mg stearate | 1.6 | 1.65 | 0.85 |
| Hardness [N] | 295 | 305 | 132 |
| Friability [%] | 0.01 | 0.01 | 0.18 |

TABLE 4

| | Active ingredient release [%] | | |
|---|---|---|---|
| Time | K.SR 160 mg | K.SR/Methocel 160/10 mg | Methocel 10 mg |
| [h] | [A] | [D] | [E] |
| 0 | 0 | 0 | 0 |
| 0.5 | 9.46 | 5.0 | 67.7 |
| 1 | 15.19 | 8.9 | 88.0 |
| 1.5 | 18.22 | 12.5 | 92.7 |
| 2 | 22.03 | 16.1 | 93.2 |
| 3 | 26.61 | 20.7 | 94.0 |
| 4 | 31.65 | 25.6 | — |
| 6 | 39.27 | 33.5 | — |
| 8 | 46.11 | 38.7 | — |
| 12 | 58.10 | 49.5 | — |
| 16 | 67.21 | 56.9 | — |

Even a small addition of Methocel® K100 M leads to a reduction in the rate of release with excellent mechanical properties. Tablets with only 10 mg of Methocel® K100 M show no release-slowing effect.

### EXAMPLE 3

Diclofenac tablets with hydroxypropylmethylcellulose (Methocel® K 100 M)

Tableting mixture (F) consisting of 200 g of diclofenac-Na and 200 g of a formulated mixture of polyvinyl acetate and polyvinylpyrrolidone in the ratio 8:2 (=Kollidon® SR), 6 g of Mg stearate; tableting mixture (G) consisting of 200 g of diclofenac and 200 g of a formulated mixture of polyvinyl acetate and polyvinylpyrrolidone in the ratio 8:2, 40 g of Methocel® K100 M and 6.0 g of Mg stearate; tableting mixture (H) consisting of 200 g of diclofenac Na and 200 g of a formulated mixture of polyvinyl acetate and polyvinylpyrrolidone in the ratio 8:2, 100 g of Methocel® K 100 M and 6.0 g of Mg stearate. Tableting mixture (I) consisting of 200 g of diclofenac Na and 200 g of Methocel® K 100 M and 6.0 g of Mg stearate.

Sieving of the individual powder ingredients through an 800 μm sieve, mixing in a Turbula mixer for 10 minutes. The respective tablets (8 mm, round, biplanar with beveled edge) were compressed in a rotary press (Korsch PH 106) under a pressure of 10 kN.

Determination of the hardness with a Krämer tablet tester (HAT-TMB), friability in an Erweka Friabilator; release test by USP XXIV method in an Erweka DT80 release apparatus, paddle method, 50 rpm, 0 to 16 h in phosphate buffer solution pH 6.8.

TABLE 5
Composition of the tablet batches [mg]:

| Batch: | F | G | H | I |
|---|---|---|---|---|
| Diclofenac Na | 100 | 100 | 100 | 100 |
| Kollidon SR | 100 | 100 | 100 | — |
| Methocel K100M | — | 20 | 50 | 100 |
| Mg stearate | 3 | 3 | 3 | 3 |
| Hardness [N] | 218 | 244 | 270 | 106 |
| Friability [%] | 0.01 | 0.01 | 0.01 | 0.15 |

TABLE 6

| | | Active ingredient release [%] | | |
|---|---|---|---|---|
| Time | K.SR | K.SR/Methocel | K.SR/Methocel | Methocel |
| [h] | 100 mg [F] | 100/20 (mg) [G] | 100/50 mg [H] | 100 mg [I] |
| 0 | 0 | 0 | 0 | 0 |
| 0.56 | 5.4 | 5.0 | 3.7 | 33.2 |
| 1 | 11.5 | 10.8 | 9.2 | 61.5 |
| 1.5 | 18.8 | 16.1 | 13.8 | 77.9 |
| 2 | 27.0 | 21.8 | 17.4 | 87.7 |
| 3 | 37.0 | 31.7 | 22.5 | 89.0 |
| 4 | 49.0 | 42.0 | 31.3 | 92.6 |
| 6.12 | 74.1 | 63.0 | 41.0 | 95.5 |
| 8 | 99.8 | 80.0 | 53.3 | 98.3 |
| 12 | 98.9 | 92.8 | 67.0 | 97.9 |
| 16 | 100.0 | 97.4 | 79.8 | 98.5 |

The release-slowing effect of a formulated mixture of polyvinyl acetate and polyvinylpyrrolidone can be increased by Methocel® K 100 M although Methocel® on its own has virtually no release-slowing effect on diclofenac. The mechanical properties of the combination are better than those of the individual components

### EXAMPLE 4

Caffeine tablets with methylhydroxyethylcellulose (Tylose® M6)

Tableting mixture (A) cf. Ex. 1. Tableting mixture (K) consisting of 320 g of caffeine and 320 g of a formulated mixture of polyvinyl acetate and polyvinylpyrrolidone in the ratio 8:2 (=Kollidon® SR), 80 g of Tylose® M6 and 3.6 g of Mg stearate.

Sieving of the individual powder ingredients through an 800 μm sieve, mixing in a Turbula mixer for 10 minutes. The respective tablets (10 mm, round, biplanar with beveled edge) were compressed in an eccentric press (Korsch EKO) under a pressure of 18 kN.

Determination of the hardness with a Kramer tablet tester (HAT-TMB), friability in an Erweka Friabilator; release test by USP XXIV method in an Erweka DT80 release apparatus, paddle method, 50 rpm, 0 to 2 h in 0.08 N HCl medium, then changed to pH 6.8 with phosphate buffer solution.

TABLE 7
Composition of the tablet batches [mg]:

| Batch: | A | K |
|---|---|---|
| Caffeine | 160 | 160 |
| Kollidon SR | 160 | 160 |
| Tylose M6 | — | 40 |
| Mg stearate | 1.6 | 1.8 |
| Hardness [N] | 295 | >350 |
| Friability [%] | 0.01 | <0.01 |

TABLE 8

Active ingredient release [%]

| Time | K.SR 160 mg | K.SR/Tylose 160/40 mg |
|---|---|---|
| [h] | [A] | [K] |
| 0 | 0 | 0 |
| 0.5 | 10.9 | 5.7 |
| 1 | 16.9 | 10.5 |
| 1.5 | 20.7 | 14.2 |
| 2 | 24.4 | 17.2 |
| 3 | 29.7 | 22.7 |
| 4 | 33.9 | 27.1 |
| 6 | 40.3 | 35.2 |
| 8 | 46.1 | 40.4 |
| 12 | 55.8 | 50.7 |
| 16 | 64.4 | 60.1 |

The small addition of Tylose reduces the rate of release and distinctly improves the mechanical properties.

### EXAMPLE 5

Caffeine tablets with stearic acid

Tableting mixture (A) cf. Ex. 1. Tableting mixture (L) consisting of 320 g of caffeine and 320 g of a formulated mixture of polyvinyl acetate and polyvinylpyrrolidone in the ratio 8:2 (=Kollidon® SR), 40 g of stearic acid and 3.6 g of Mg stearate.

Sieving of the individual powder ingredients through an 800 μm sieve, mixing in a Turbula mixer for 10 minutes. The respective tablets (10 mm, round, biplanar with beveled edge) were compressed in an eccentric press (Korsch EKO) under a pressure of 18 kN.

Determination of the hardness with a Kramer tablet tester (HAT-TMB). friability in an Erweka Friabilator; release test by USP XXIV method in an Erweka DT80 release apparatus, paddle method, 50 rpm, 0 to 2 h in 0.08 N HCl medium, then changed to pH 6.8 with phosphate buffer solution.

TABLE 9
Composition of the tablet batches [mg]:

| Batch: | A | L |
|---|---|---|
| Caffeine | 160 | 160 |
| Kollidon SR | 160 | 160 |
| Stearic acid | — | 40 |
| Mg stearate | 1.6 | 1.8 |
| Harndess [N] | 295 | 274 |
| Friability [%] | 0.01 | 0.02 |

TABLE 10

Active ingredient release [%]

| Time | K.SR 160 mg | K.SR/stearic acid 160/40 mg |
|---|---|---|
| [h] | [A] | [L] |
| 0 | 0 | 0 |
| 0.5 | 10.9 | 7.3 |
| 1 | 16.9 | 11.5 |
| 1.5 | 20.7 | 14.8 |
| 2 | 24.4 | 17.2 |
| 3 | 29.7 | 21.8 |
| 4 | 33.9 | 24.7 |
| 6 | 40.3 | 30.0 |
| 8 | 46.1 | 34.4 |
| 12 | 55.8 | 43.4 |
| 16 | 64.4 | 49.7 |

The small addition of stearic acid distinctly reduces the rate of release of the active ingredient.

### EXAMPLE 6

Propranolol tablets with methacrylic acid/ethyl acrylate copolymer (Kollicoat® MAE 100 P)

Tableting mixture (M) consisting of 320 g of Propranolol HCl and 320 g of a formulated mixture of polyvinyl acetate and polyvinylpyrrolidone in the ratio 8:2 (=Kollidon® SR), and 6.4 g of Mg stearate; tableting mixture (N) consisting of 320 g of propranolol HCl and 320 g of a formulated mixture of polyvinyl acetate and polyvinylpyrrolidone in the ratio 8:2, 80 g of Kollicoat® MAE 100 P and 7.2 g of Mg stearate.

Sieving of the individual powder ingredients through an 800 μm sieve, mixing in a Turbula mixer for 10 minutes. The respective tablets (10 mm, round, biplanar with beveled edge) were compressed in a rotary press (Korsch PH 106) under a pressure of 18 kN.

Determination of the hardness with a Kramer tablet tester (HAT-TMB), friability in an Erweka Friabilator; release test by USP XXIV method in an Erweka DT80 release apparatus, paddle method, 50 rpm, 0 to 2 h in 0.08 N HCl medium, then changed to pH 6.8 with phosphate buffer solution.

TABLE 11
Composition of the tablet batches [mg]:

| Batch: | M | N |
|---|---|---|
| Propranolol | 160 | 160 |
| Kollidon SR | 160 | 160 |
| Kollicoat MAE 100 P | — | 40 |
| Mg stearate | 3.2 | 3.6 |
| Hardness [N] | 216 | 271 |
| Friability [%] | 0.02 | 0.02 |

TABLE 12

| | Active ingredient release [%] | |
|---|---|---|
| Time | K.SR 160 mg | K.SR/K. MAE 160/40 mg |
| [h] | [M] | [N] |
| 0 | 0 | 0 |
| 0.5 | 19.4 | 10.0 |
| 1 | 25.3 | 15.5 |
| 1.5 | 31.8 | 18.9 |
| 2 | 38.0 | 22.4 |
| 2.5 | 41.5 | 24.6 |
| 3 | 45.8 | 26.5 |
| 4 | 53.9 | 30.6 |
| 5 | 59.7 | 33.4 |
| 6 | 64.2 | 34.7 |
| 7 | 68.9 | 36.6 |
| 8 | 71.8 | 38.2 |
| 9 | 74.8 | 40.4 |
| 10 | 77.3 | 41.7 |
| 11 | 79.4 | 43.7 |
| 12 | 81.8 | 45.4 |
| 16 | 86.3 | 51.7 |

The addition of Kollicoat® MAE 100P improves the mechanical properties and reduces the release.

We claim:

1. An oral dosage form with delayed release of active ingredient and high mechanical stability, comprising
a) one or more active ingredients
b) a formulated mixture of polyvinyl acetate and polyvinylpyrrolidone
c) water-soluble polymers or low or high molecular weight lipophilic additives
d) and other conventional excipients.

2. An oral dosage form as claimed in claim 1, wherein the ratio of polyvinyl acetate to polyvinylpyrrolidone is from 6:4 to 9:1.

3. An oral dosage form as claimed in either of claims 1 or 2, wherein a formulated mixture of polyvinyl acetate and polyvinylpyrrolidone in the ratio 8:2 is employed.

4. An oral dosage form as claimed in any of claims 1 to 3, which is a tablet, extrudate, pellet or granulate.

5. An oral dosage form as claimed in any of claims 1 to 4, wherein a water-soluble or water-insoluble release-delaying coating is applied to the oral dosage form.

6. An oral dosage form as claimed in any of claims 1 to 5, wherein the water-soluble or lipophilic polymers are selected from the group of: polyvinyl alcohols, polyethylene glycols, polyoxyethylene/polyoxypropylene block copolymers, polyvinylpyrrolidones and derivatives, vinyl acetate/vinylpyrrolidone copolymers, preferably polyethylene glycols, polyvinylpyrrolidones, vinyl acetate/vinylpyrrolidone copolymers or maltodextrins, and salts thereof.

7. An oral dosage form as claimed in any of claims 1 to 6, wherein the water-soluble swelling polymers are selected from the group of: alginates, pectins, galactomannans, carrageenans, dextran, curdlan, pullulan, gellan, chitin, gelatin, xanthans, hemicelluloses, cellulose derivatives such as methylcellulose, hydroxypropylmethylcellulose, hydroxypropylcellulose, hydroxyethylcellulose, methylhydroxyethylcellulose, carboxymethylcellulose, starch derivatives such as carboxymethyl starch, degraded starch, polyacrylic acid, polymethacrylic acid, acrylic acid/methacrylic acid copolymers, and salts thereof.

8. An oral dosage form as claimed in any of claims 1 to 6, wherein the lipophilic additives are selected from the group of: cellulose derivatives such as ethylcellulose, cellulose acetate, cellulose acetate phthalate, cellulose acetate succinate, hydroxypropylmethylcellulose acetate phthalate, hydroxypropylmethylcellulose acetate succinate, acrylic ester/methacrylic ester copolymers, in particular methyl methacrylate/ethyl acrylate copolymers, ammoniomethacrylate copolymer type A and type B, methacrylic acid/acrylic ester copolymers, in particular methacrylic acid/ethyl acrylate copolymers, fatty alcohols such as stearyl alcohol, fatty acids such as stearic acid, fatty acid esters and fatty alcohol esters, glycerides, waxes, lecithin.

9. An oral dosage form as claimed in any of claims 1 to 7, which is produced by direct compression, extrusion, melt extrusion, pelleting, compaction, wet granulation.

10. An oral dosage form as claimed in any of claims 1 to 8, wherein binders, extenders/fillers, disintegrants, lubricants, flow regulators, dyes, stabilizers such as antioxidants, wetting agents, preservatives, release agents, flavorings and sweeteners are employed as conventional excipients.

11. An oral dosage form as claimed in any of claims 1 to 9, wherein the formulated mixture of polyvinyl acetate and polyvinylpyrrolidone is present in a proportion of from 10 to 80% based on the total weight of the tablet.

12. An oral dosage form as claimed in any of claims 1 to 10, wherein the water-soluble polymers and/or the lipophilic additives are present in a proportion of from 1 to 40% based on the total weight of the tablet.

13. An oral dosage form as claimed in any of claims 1 to 11, wherein hydroxypropylmethylcelluloses are employed as water-soluble polymers.

14. An oral dosage form as claimed in any of claims 1 to 12, wherein polyvinylpyrrolidones or vinyl acetate/vinylpyrrolidone copolymers are employed as water-soluble polymers.

15. An oral dosage form as claimed in any of claims 1 to 14, which is a press-coated tablet whose core is rich in active ingredient.

16. An oral dosage form as claimed in any of claims 1 to 15, which comprises as active ingredients food supplements or additives, vitamins, minerals or trace elements or active pharmaceutical ingredients.

17. An oral dosage form as claimed in any of claims 1 to 16, which comprises active pharmaceutical ingredients as active ingredients.

18. A dosage form as claimed in any of claims 1 to 17, wherein the active pharmaceutical ingredient is selected from the group of benzodiazepines, antihypertensives, vitamins, cytostatics, anesthetics, neuroleptics, antidepressants, antibiotics, antimycotics, fungicides, chemotherapeutics, urologicals, platelet aggregation inhibitors, sulfonamides, spasmolytics, hormones, immunoglobulins, sera, thyroid therapeutics, psychopharmaceuticals, antiparkinson agents and other antihyperkinetics, ophthalmologicals, neuropathy products, calcium metabolism regulators, muscle relaxants, lipid-lowering agents, liver therapeutics, coronary agents, cardiac agents, immunotherapeutics, regulatory peptides and their inhibitors, hypnotics, sedatives, gynecologicals, antigout agents, fibrinolytics, enzyme products and transport proteins, enzyme inhibitors, emetics, perfusion promoters, diuretics, diagnostics, corticoids, cholinergics, biliary therapeutics, antiasthmatics, bronchospasmolytics, beta-receptor blockers, calcium channel blockers, ACE inhibitors, arteriosclerosis remedies, antiinflammatory agents, anticoagulants, antihypotensives, antihypoglycemics, antifibrinolytics, antiepileptics, antiemetics, antidotes, antidiabetics, antiarrhythmics, antianemics, antiallergics, anthelmintics, analgesics, analeptics, aldosterone antagonists, weight-reducing agents.

19. A drug for delayed release of active ingredient, which is an oral dosage form as claimed in any of claims 1 to 18.

20. The use of the oral dosage forms as claimed in any of claims 1 to 17 for producing drugs with delayed release of active ingredient.

21. The use of the oral dosage forms as claimed in any of claims 1 to 17 for delayed release of active ingredients which are food supplements or additives, vitamins, minerals or trace elements.
